(19) United States
(12) Patent Application Publication
(10) Pub. No.: US 2025/0243503 A1
(43) Pub. Date: Jul. 31, 2025

(54) HD-ZIP TRANSCRIPTION FACTOR ARHDZ19 OF ANOECTOCHILUS ROXBURGHII HONGXIA, ENCODING GENE AND USE OF ENCODING GENE

(71) Applicant: Zhejiang A&F University, Hangzhou (CN)

(72) Inventors: Qingsong SHAO, Hangzhou (CN); Yu ZHANG, Hangzhou (CN); Taowei ZHONG, Hangzhou (CN); Aimin LV, Hangzhou (CN)

(73) Assignee: Zhejiang A&F University, Hangzhou (CN)

(21) Appl. No.: 18/888,245

(22) Filed: Sep. 18, 2024

(30) Foreign Application Priority Data
Jan. 26, 2024 (CN) 2024101119100

(51) Int. Cl. C12N 15/82; C07K 14/415

(52) U.S. Cl. CPC C12N 15/8267; C07K 14/415; C12N 15/8205

(57) ABSTRACT

An HD-ZIP transcription factor ArHDZ19 of Anoectochilus roxburghii ‘Hongxia’, encoding gene, and use of encoding gene are provided. The transcription factor ArHDZ19 includes a polypeptide having the amino acid sequence as shown in SEQ ID NO: 2. A gene for encoding the above-mentioned proteins having the nucleotide sequence as shown in SEQ ID NO: 1 is provided. The present invention provides a theoretical basis for using genetic engineering technology to promote the reproductive growth of Anoectochilus roxburghii ‘Hongxia’ and improve seed vitality, and has important application value.

### CROSS REFERENCE TO THE RELATED APPLICATIONS

This application is based upon and claims priority to Chinese Patent Application No. 2024101119100, filed on Jan. 26, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBJSQT007_SequenceListing.xml, created on Sep. 18, 2024, and is 6,180 bytes in size.

### TECHNICAL FIELD

The present invention belongs to the technical field of genetic engineering application, and relates to an important homeodomain-leucine zipper (HD-ZIP) protein in the reproductive growth process of Anoectochilus roxburghii, and in particular to an HD-ZIP transcription factor ArHDZ19 of Anoectochilus roxburghii ‘Hongxia’, an encoding gene of the ArHDZ19, and a use of the encoding gene.

### BACKGROUND

Anoectochilus roxburghii (Anoectochilus roxburghii (Wall.) Lindl.), also known as Jinsicao and Jinxianlan in Chinese, is a perennial herb of the genus Anoectochilus of the Orchidaceae family and also a rare and precious Chinese medicinal material. It tastes sweet and has a neutral nature, and the whole plant of Anoectochilus roxburghii can be used for medicinal purpose, with the effects of clearing heat, cooling blood, removing dampness and toxic material, thereby earning the reputation of “King of Medicine”. In recent years, the wide application of Anoectochilus roxburghii in many fields has led to an increasing demand in the Chinese and international markets. However, the pollen and stigma of Anoectochilus roxburghii are viable for a short period of time, and the natural reproduction rate of seeds is low. The seeds of Anoectochilus roxburghii have embryo abortion after fertilization, resulting in reproductive disorders in Anoectochilus roxburghii.

Homeodomain-leucine zipper (HD-ZIP) proteins are a class of transcription factors unique to higher plants. They participate in plant-specific biological processes and play an important role in the growth and development of higher plants. HD-ZIP III subfamily transcription factors participate in regulating the reproductive development of plants and are closely related to the embryonic and post-embryonic morphogenesis, cell differentiation, lateral organogenesis, vascular cell division, and polarity establishment of plants. However, there are no reports on the involvement of HD-ZIP proteins in the reproductive development of Anoectochilus roxburghii.

### SUMMARY

In order to fill the gaps in the research on the cloning and expression pattern of the ArHDZ19 gene of Anoectochilus roxburghii ‘Hongxia’ and the transcription factor ArHDZ19 of Anoectochilus roxburghii ‘Hongxia’, the present invention provides an ArHDZ19 gene sequence of a HD-ZIP of Anoectochilus roxburghii ‘Hongxia’ and the encoded amino acid sequence, protein subcellular localization, and transgenic plant growth phenotype thereof, thereby providing a theoretical basis for using genetic engineering technology to regulate the expression of the ArHDZ19 gene and improve the breeding efficiency of Anoectochilus roxburghii in the future.

In one aspect, the present invention provides an HD-ZIP transcription factor ArHDZ19 of Anoectochilus roxburghii ‘Hongxia’, having the function of promoting reproductive development and improving seed traits. The transcription factor ArHDZ19 includes a polypeptide (protein) having an amino acid sequence as shown in SEQ ID NO: 2; or a protein having the characteristics of the HD-ZIP transcription factor ArHDZ19 of roxburghii ‘Hongxia’ and obtained by replacement, deletion or addition of one or more amino acids on the amino acid sequence shown in SEQ ID NO: 2.

In another aspect, the present invention provides an encoding gene for encoding the HD-ZIP transcription factor ArHDZ19 of Anoectochilus roxburghii ‘Hongxia’, and the nucleotide sequence of the encoding gene is specifically: (a) a base sequence from positions 1 to 2553 as shown in SEQ ID NO: 1; or (b) a sequence having at least 70% homology with nucleic acids at positions 1 to 2553 as shown in SEQ ID NO: 1.

In the present invention, the terms “isolated DNA” and “purified DNA” mean that the DNA or fragment has been isolated from the sequences located on two sides of it in a natural state, and also mean that the DNA or fragment has been separated from the components accompanying the nucleic acid in the natural state, and has been separated from the proteins accompanying it in the cell.

In the present invention, the encoding gene of the HD-ZIP transcription factor ArHDZ19 of Anoectochilus roxburghii ‘Hongxia’ refers to a nucleotide sequence that encodes a polypeptide having the protein activity of the Anoectochilus roxburghii ‘Hongxia’, such as the nucleotide sequence from positions 1 to 2553 shown in SEQ ID NO: 1 and its degenerate sequence. The degenerate sequence refers to a sequence generated after one or more codons in nucleotides at positions 1 to 2553 shown in SEQ ID NO: 1 are replaced by degenerate codons encoding the same amino acid. Due to the degeneracy of codons, a degenerate sequence with a homology of as low as about 70% with the nucleotide sequence from positions 1 to 2553 shown in SEQ ID NO: 1 can also be encoded to obtain the sequence shown in SEQ ID NO: 2. The described encoding gene may also refer to a nucleotide sequence having at least 70% homology with the nucleotide sequence shown in SEQ ID NO: 1.

The described encoding gene may also refer to being capable of encoding a variant form of the sequence shown in SEQ ID NO: 1 that has the same function as the natural HD-ZIP transcription factor ArHDZ19 of Anoectochilus roxburghii ‘Hongxia’, and these variant forms include (but are not limited to): usually a deletion, insertion and/or substitution of 1 to 120 nucleotides, and an addition of less than 120 nucleotides at the 5′ and/or 3′ ends.

In the present invention, real-time fluorescence quantitative PCR may be used to analyze the expression pattern of the product of the ArHDZ19 gene of Anoectochilus roxburghii ‘Hongxia’, that is, to analyze the presence and quantity of the mRNA transcripts of the ArHDZ19 gene of Anoectochilus roxburghii ‘Hongxia’ in the cells.

In addition, according to the nucleotide sequence and amino acid sequence of the ArHDZ19 gene of Anoectochilus roxburghii ‘Hongxia’ of the present invention, homologous genes or homologous proteins related to the ArHDZ19 gene of Anoectochilus roxburghii ‘Hongxia’ can be screened on the basis of the homology of nucleic acids or the homology of expressed proteins.

The relevant full-length nucleotide sequence of the ArHDZ19 gene of Alocasia roxburghii ‘Hongxia’ of the present invention or fragments thereof can usually be obtained by means of PCR amplification, recombination or artificial synthesis. For PCR amplification, primers may be designed according to the relevant nucleotide sequence disclosed in the present invention, and a commercially available cDNA bank or a cDNA bank prepared by a conventional means known to those skilled in the art can be used as a template for amplification to obtain a relevant sequence. When the sequence is long, PCR amplification may be performed twice or more, and then the fragments obtained after the amplifications are spliced together in a correct order.

Once the relevant sequence is obtained, a large quantity of the relevant sequence may be obtained by means of recombination. This is usually done by cloning the relevant sequence into a vector, and then transferring it into a cell, and finally isolating the relevant sequence from proliferated host cells by conventional methods.

In addition, mutations may also be introduced into the protein sequence of the present invention by chemical synthesis.

In addition to being produced by means of recombination, fragments of the protein of the present invention may also be produced by direct peptide synthesis using a solid phase technology. Each fragment of the protein of the present invention can be chemically synthesized separately and then chemically linked to produce a full-length molecule.

The present invention further provides a recombinant expression vector, which includes the encoding gene of the described HD-ZIP transcription factor ArHDZ19 of Anoectochilus roxburghii ‘Hongxia’. The described recombinant expression vector is pHB-ArHDZ19.

The present invention further provides a use of the encoding gene of the HD-ZIP transcription factor ArHDZ19 of Anoectochilus roxburghii ‘Hongxia’ in promoting the reproductive growth of Anoectochilus roxburghii and improving the seed vitality.

The use described above includes: constructing a recombinant expression vector including the described encoding gene of the described transcription factor ArHDZ19, transforming a plant host, and performing cultivation and screening to obtain a transgenic plant.

As a rare and precious Chinese medicinal material, Anoectochilus roxburghii has a large market demand. In the present invention, the encoding sequence of the important regulatory transcription factor ArHDZ19 in the growth and development process of Anoectochilus roxburghii ‘Hongxia’ is cloned for the first time, the expression pattern of the ArHDZ19 gene is analyzed by means of real-time fluorescence quantitative PCR, and the subcellular localization of the transcription factor ArHDZ19 is analyzed by transient expression in epidermal cells of tobacco leaves, thereby laying a theoretical basis for regulating the spatiotemporal expression of the ArHDZ19 gene by genetic engineering technology in the future to improve the vitality of Anoectochilus roxburghii seeds, promote reproductive growth, and breeding new varieties, and also achieving a great application value.

### BRIEF DESCRIPTION OF THE DRAWINGS

FIGS. 1A-1B shows the homology comparison (DNAMAN) between an HD-ZIP gene of Anoectochilus roxburghii ‘Hongxia’ and an HD-ZIP protein sequence of Arabidopsis thaliana and the evolutionary tree analysis of homologous genes. (FIG. 1A) Diagram of evolutionary tree analysis of ArHDZ19 protein and homologous proteins in other species. (FIG. 1B) Diagram of alignment of multiple sequences.

FIG. 2 is a localization diagram of the transcription factor ArHDZ19 of Anoectochilus roxburghii ‘Hongxia’ in the epidermal cells of tobacco leaves.

FIG. 3 shows expression changes of the ArHDZ19 gene of Anoectochilus roxburghii ‘Hongxia’ during the growth and development of plants.

FIG. 4 shows growth and development phenotypes of wild-type and OE-ArHDZ19 plants.

FIGS. 5A-5D show seed traits of the wild-type and OE-ArHDZ19 plants. (FIG. 5A) Schematic diagram of the seed traits of the wild-type plant. (FIG. 5B) Schematic diagram of the seed traits of the OE-ArHDZ19 plant. (FIG. 5C) Schematic diagram of hundred-grain weight of seeds. (FIG. 5D) Schematic diagram of seed length.

### DETAILED DESCRIPTION OF THE EMBODIMENTS

The present invention will be further described below with reference to specific examples. It should be understood that these examples are only for illustrating the present invention and are not intended to limit the scope of the present invention.

Experimental methods without specific conditions listed in the following examples are generally conducted under conventional conditions, such as those described in Molecular Cloning: A Laboratory Manual (Fourth Edition), or the conditions suggested in the reagent instructions.

### Example 1 Cloning of ArHDZ19 gene of Anoectochilus roxburghii ‘Hongxia’

### 1. Acquisition of Plant Materials

Tissues, such as stems, leaves, buds, and flowers, of normally growing Anoectochilus roxburghii ‘Hongxia’ plants were taken to extract total RNA.

### 2. Extraction of RNA

The total RNA was extracted using a “TransZol Up Plant Total RNA Extraction Kit” (Beijing TransGen Biotech Co., Ltd), and the integrity of the RNA was identified by gel electrophoresis. The purity and concentration of the RNA were determined by a spectrophotometer (Nanodrop 2000).

### 3. Full-Length Cloning of the Gene

Based on the nucleotide sequence and protein function annotation results provided by the laboratory's previous full-length transcriptome analysis, the full-length ArHDZ19 gene of Anoectochilus roxburghii ‘Hongxia’ was obtained. The extracted RNA was reverse transcribed (TransScript One-Step gDNA Removal and cDNA Synthesis SuperMix) to obtain cDNA. A first-strand cDNA, used as a template, was subjected to PCR amplification using primers ArHDZ19-F (5′-ATGGCGATGATGGTGAGCGC-3′) and ArHDZ19-R (5′-TCAAACAAAAGACCAGTTGATGATCATAA-3′) to obtain a 2553bp fragment. The fragment was recovered and linked to a pMD19-T vector and then delivered to Hangzhou Youkang for sequencing with M13-47 and RV-M as universal primers.

By combining the sequencing results with the prediction from the ORF Finding of NCBI (http://www.ncbi.nlm.nih.gov/gorf), the ORFs (Open Reading Frames) of the ArHDZ19 gene of Anoectochilus roxburghii were found, and then the full-length encoding sequence with a length of 2553 bp (SEQ ID NO: 1) was obtained by amplification. The sequencing results were compared with the database (GenBank, http://blast.ncbi.nlm.nih.gov/) using BLAST on the NCBI website. Its nucleotide sequence and encoding protein had a high homology with the known DcHD-Zip III (XP_020698923.1) nucleotide and protein sequences of Dendrobium officinale. So it was initially considered to be an HD-ZIP gene.

### Example 2 Sequence Information and Homology Analysis of the ArHDZ19 Gene of Anoectochilus roxburghii ‘Hongxia’

The full-length ORF sequence of the ArHDZ19 gene of Anoectochilus roxburghii ‘Hongxia’ of the present invention is 2553bp. For the detailed sequence, please refer to the sequence shown in SEQ ID NO: 1. The amino acid sequence of the transcription factor ArHDZ19 protein of Anoectochilus roxburghii ‘Hongxia’, with a total of 850 amino acids, a molecular weight of 93.4 kDa, and an isoelectric point (pI) of 8.19, was deduced from the ORF sequence. For the detailed sequence, please refer to the sequence shown in SEQ ID NO: 2.

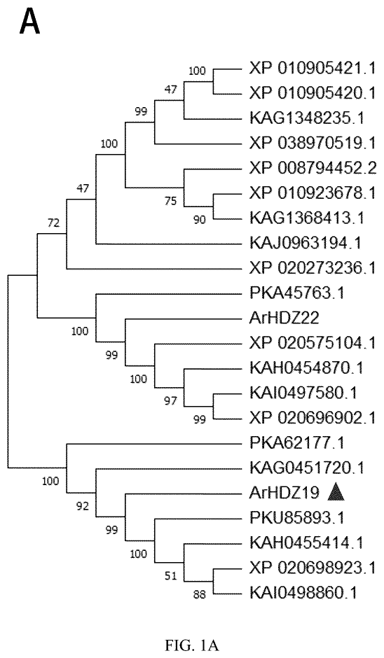

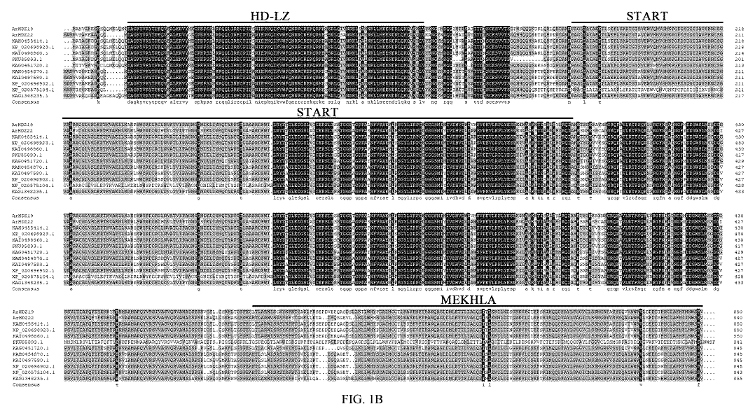

The ORF sequence of the ArHDZ19 gene of Anoectochilus roxburghii ‘Hongxia’ and the amino acid sequence of its encoding protein were searched for nucleotide and protein homology in NCBI using the BLAST program. The results showed that the transcription factor ArHDZ19 of Anoectochilus roxburghii ‘Hongxia’ had a very high homology with DcHD-Zip III (XP_020698923.1) of Dendrobium officinale at the amino acid level, as shown in FIG. 1B. Evolutionary tree analysis showed that the transcription factor ArHDZ19 of Anoectochilus roxburghii ‘Hongxia’ had a high homology with HD-ZIP proteins of other known species, as shown in FIG. 1A.

### Example 3 Construction of Recombinant Expression Vector pHB-ArHDZ19 of Anoectochilus roxburghii and Analysis of Subcellular Localization of the Transcription Factor ArHDZ19 in Tobacco Leaves

Specific primers ArHDZ19-F (5′-TCGGTACCCGGGGATCCATGGCGATGGTGGGGAAGGA-3′) and ArHDZ19-R (5′-TGCTCACCATGTCGACGACAAATGACCAGTTCATGAAC-3′) were designed respectively from the start codon and the stop codon, and BamHI and SpelI restriction sites were introduced on two sides of the full-length gene sequence. The target fragment plasmid with restriction sites and the pHB binary transformation vector were double-digested with BamHI and SpelI, the pHB vector after restriction digestion and the ArHDZ19 fragment were recovered and linked by T4 ligase at 16° C. for 6 h to construct the recombinant expression vector pHB-ArHDZ19.

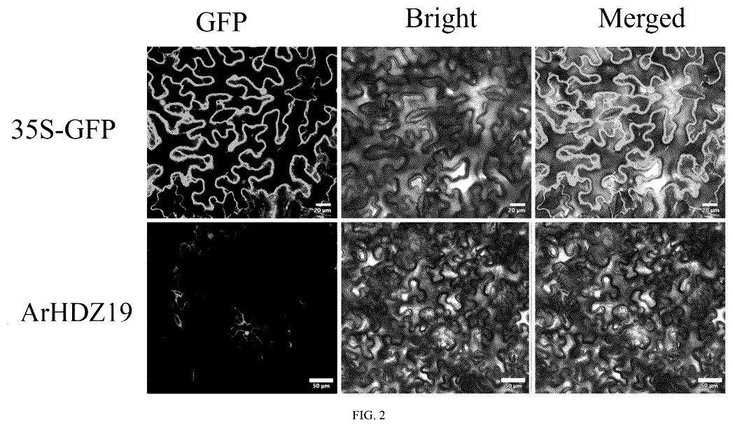

The recombinant expression vector pHB-ArHDZ19 which was identified to be correct was transformed into Agrobacterium EHA105. The identified EHA105 strain was inoculated in 5 mL of YEP (containing 50 mg/L Kan), and cultured at 28° C. and 180 rpm until OD₆₀₀ was about 0.6; 1 mL of the culture solution was taken and added to 25 mL of YEP liquid medium, and cultured at 28° C. until OD₆₀₀ was about 0.6; 10 mL of the culture solution was taken and centrifuged at 4500 rpm for 15 min; the bacteria were then suspended in an MS liquid medium until OD₆₀₀ was about 0.6, AS and MES were then added, and the suspension was then placed at room temperature for more than 3 h; then, the suspension was injected into tobacco leaves, cultured in the dark for 48 h, and observed under a laser confocal microscope at an excitation wavelength of 514 nm, as shown in FIG. 2.

### Example 4 Expression Changes of ArHDZ19 Gene of Anoectochilus roxburghii in Different Tissues of Anoectochilus roxburghii

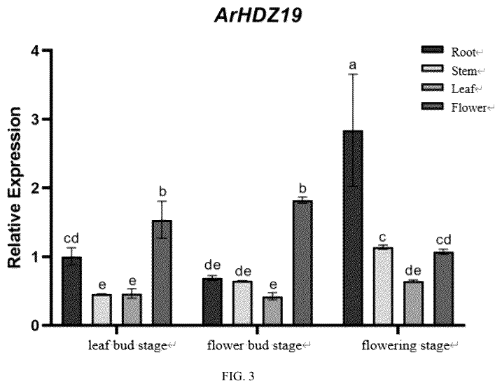

1. Acquisition of materials: Root, stem, leaf and flower tissue samples at different developmental stages (leaf bud stage, flower bud stage and flowering stage) during the growth of Anoectochilus roxburghii plants were taken, each 0.1 g. The samples were wrapped with aluminum platinum paper separately and put into liquid nitrogen, then transferred to an −80° C. ultra-low temperature refrigerator for storage.
2. The extraction of RNA, the integrity, purity and concentration determination of RNA and the acquisition of cDNA were performed with reference to Example 1.
3. Specific primers were designed to perform real-time fluorescence quantitative PCR to analyze the expression of the gene in various tissues. The specific primers, designed for quantitative analysis of ArHDZ19 gene in Real-time PCR according to the obtained ArHDZ19 gene sequence of Anoectochilus roxburghii, included the primer qArHDZ19-F (5′-GTATACGCCGGAGCAAGTGG-3′) and the primer qArHDZ19-R (5′-TCTCTGATGAGCTGCTGCCT-3′), as well as the primers for the internal reference gene actin which were actin-F (5′-GCTAGTGGCCGTACAACTGG-3′) and actin-R (5′-GCCAGCAAGGTCCAATCGAA-3′).
4. Standard curves of the target gene and the internal reference gene: The standard cDNA solution was diluted with ddH₂O in a gradient manner; the diluted cDNA, used as a template, was subjected to real-time PCR amplification using the specific primers of the target gene and the internal reference gene, and then a melting curve and a standard curve were plotted. The melting curve was analyzed to determine whether the melting curve of the target gene and the internal reference gene has a single peak, so as to determine whether a single PCR amplification product can be obtained using the primers; and the appropriate dilution multiple of the template cDNA was determined according to the standard curve.
5. Real-time fluorescence quantitative analysis of the target gene in the samples to be tested: The first strand of synthesized cDNA, used as a template, was subjected to amplification using specific primers of the target gene and the internal reference gene to conduct fluorescence quantitative analysis. Real-time PCR was performed using a Bio-Rad CFX real-time fluorescence quantification instrument, where the reaction system was 20 μL, and the reaction procedure was: pre-denaturation at 94° C. for 20 s, 94° C. for 15 s; 55° C. for 15 s; 72° C. for 15 s; 40 cycles.
6. The 2^{-ΔΔCt} method was used for relative quantitative analysis. The results showed that the expression level of the ArHDZ19 gene of Anoectochilus roxburghii increased significantly in Anoectochilus roxburghii tissues at different development stages, as shown in FIG. 3.

### Example 5 ArHDZ19 Gene Transformation of Arabidopsis thaliana

### 1. Construction of Plant Expression Vector

For the introduced restriction sites, please refer to Example 3. The target fragment plasmid with restriction sites and the pHB binary transformation vector were double digested with BamHI and SpeI, the vector after restriction digestion was recovered and linked by means of homologous recombination, and the recombinant expression vector was then transformed into Agrobacterium EHA105.

2. Transformation of Arabidopsis thaliana

Transformation of Arabidopsis thaliana by flower infection: The operation of infecting Arabidopsis thaliana by Agrobacterium-mediated inflorescence immersion was specifically performed as follows.
\textcircled{1} When wild-type Arabidopsis thaliana grew to the middle and early flowering period, the best period for infection of Arabidopsis thaliana was reached. The blooming flowers and fruit pods were cut off one day before infection, and the flowers in buds were remained and were given plenty of water and fertilizer to get ready for infection.
\textcircled{2} 100 μL of positive Agrobacterium culture was taken and expanded in 25 mL of LB liquid medium containing Kana (50 mg/L) and Rif (25 mg/L). The culture was then shaken in a constant-temperature shaker (28° C., 220 rpm) until the OD₆₀₀ of the culture reached 0.8-1.0.
\textcircled{3} When the OD₆₀₀ of the expanded Agrobacterium culture reached 0.8-1.0, centrifugation was performed at 6000 rpm for 10 min to collect the bacteria, and the supernatant was discarded.
\textcircled{4} The bacteria were suspended with an MS resuspension solution (4.43 g/L MS powder+20 g/L sucrose, pH 5.8) and the OD₆₀₀ was adjusted to 0.8-1.0. Then, Tween-20 was added to control the final concentration at 0.02%, and the suspension was then well shaken, thus obtaining the infection solution.
\textcircled{5} Inflorescences of each Arabidopsis thaliana plant was soaked completely in the infection solution for 10 min.
\textcircled{6} After the infection, the Arabidopsis thaliana plants were bagged and placed flat in a dark room for dark culture for 1 day to maintain humidity. After the dark culture, the Arabidopsis thaliana plants were placed in a greenhouse with a temperature of 22° C. and a humidity of 65% to be cultured normally for 16 h with light and 8 h in the dark each day.
\textcircled{7} One week later, the described operations were repeated to infect Arabidopsis thaliana again to increase the success rate of transformation.
\textcircled{8} After the second infection, the Arabidopsis thaliana was cultured normally and managed in the conventional cultivation method until harvest.

### 3. Screening of Positive Transgenic Lines

Genomic DNA and RNA were extracted using the kit (Beijing TransGen Biotech Co., Ltd) to identify positive plants.

### Example 6 Phenotypic Analysis of OE-ArHDZ19 Arabidopsis thaliana

The objects selected for phenotypic observation and measurement of Arabidopsis thaliana were positive homozygous T3 plants and positive homozygous T3 seeds (OE), and Arabidopsis thaliana transformed with pHB empty vector was used as negative control (CK). The indicators for phenotypic observation and measurement were: plant growth cycle, plant height, seed length and width, and hundred-grain weight of seeds.

Method for observing plant growth cycle: Three Arabidopsis thaliana plants were randomly selected from transgenic plants on day 37 after sowing, and the difference in growth cycle between the selected plants and control plants was observed and recorded in the form of photos. Method for measuring plant height: On day 44 after sowing, three Arabidopsis thaliana plants were randomly selected from control plants and three Arabidopsis thaliana plants were randomly selected from transgenic plants, and these selected plants were taken out of pots with the cultivation medium washed off, and then placed on black cloth to measure the distance from the base of the rosette leaf to the top of the inflorescence and photographed, as shown in FIG. 4.

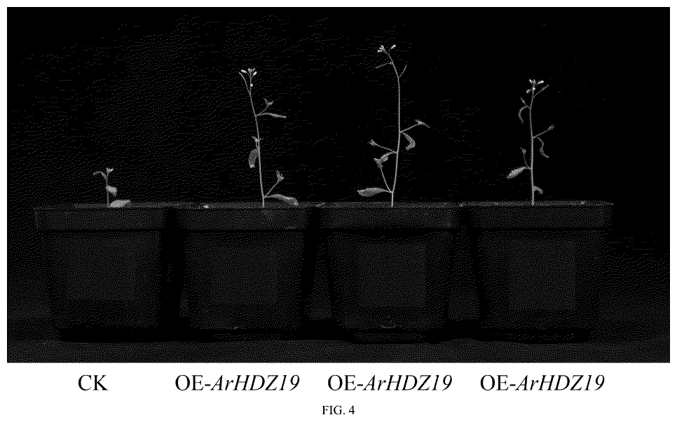

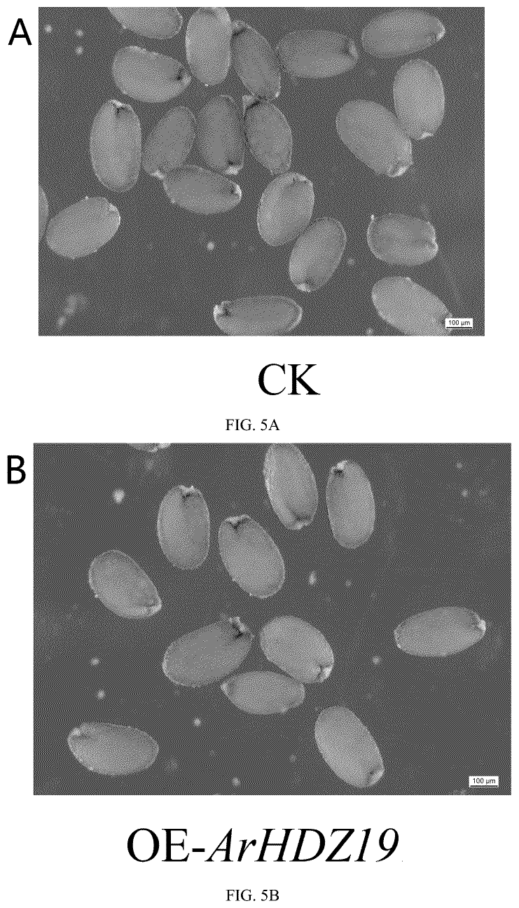

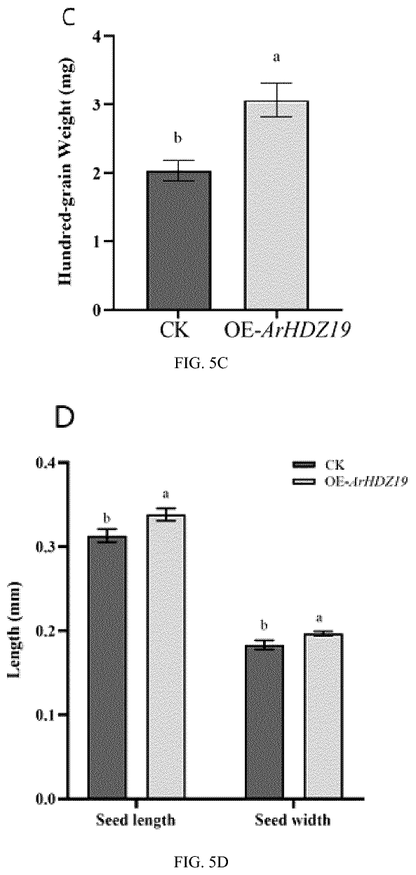

Method for measuring seed length and width: Several control plant seeds and transgenic plant seeds were selected and randomly spread on a glass slide, and then observed under an optical microscope and photographed under natural light. Then, ten seeds were randomly selected from the field of view to calculate the average length and width using a scale. Method for measuring hundred-grain weight of seeds: 100 seeds from the control plants and 100 seeds from transgenic plants were weighed on an analytical balance respectively, as shown in FIGS. 5A-5D.

The above describes the specific embodiments of the invention. It should be understood that the invention is not limited to the above specific embodiments, those skilled in the art can make various modifications or changes within the scope of the claims, and these modifications or changes do not affect the essence of the invention.

What is claimed is:

1. An HD-ZIP transcription factor ArHDZ19 of Aromatica roxburghii ‘Hongxia’, comprising a polypeptide having the amino acid sequence as shown in SEQ ID NO: 2.

2. An encoding gene for encoding the HD-ZIP transcription factor ArHDZ19 of the Anoectochilus roxburghii ‘Hongxia’ according to claim 1, comprising the nucleotide sequence as shown in SEQ ID NO: 1.

3. A recombinant expression vector, comprising the encoding gene for encoding the HD-ZIP transcription factor ArHDZ19 of the Anoectochilus roxburghii ‘Hongxia’ according to claim 2.

4. The recombinant expression vector according to claim 3, wherein the recombinant expression vector is pHB-ArHDZ19.

5. A method for promoting a reproductive growth of the Anoectochilus roxburghii ‘Hongxia’ and improving a seed vitality, comprising using the encoding gene for encoding the HD-ZIP transcription factor ArHDZ19 of the Anoectochilus roxburghii ‘Hongxia’ according to claim 2.

6. The method according to claim 5, wherein the method comprises a construction of a recombinant expression vector comprising the encoding gene for encoding the HD-ZIP transcription factor ArHDZ19, a transformation of a plant host, and a cultivation and a screening of a transgenic plant.


SEQUENCE LISTING (2 sequences)

SEQ ID NO: 1 - DNA, 2553 bp, synthetic construct
  source 1..2553: mol_type = other DNA
atggcgatggtggggaaggaaggaaaatcccagcagcatgagctgcagcatattgatgcaggtaagtacgttaggtatacgccggagcaagtggaggctcttgagagggtttatatggagtgtccaaagccgagctcactgaggaggcagcagctcatcagagagtgcccgattctctctaacatcgagcccaagcagatcaaagtctggttccagaaccggaggtgtcgtgagaagcagaggaaagaggcttctcgtctccagacagttaacagaaagctttctgctatgaacaagttgctgatggaggagaatgaccgattgcaaaagcaagtgtcccacttggtttatgagaatggatgcatgcgtcaacagattcatactccatctgcgacaactactgacactagctgcgagtctgtagtaactagtggcccgcacaaacaacaacaaaatacaaaattgcagcatcttcaaagagatgctaacaacccagcaggtctacttgctatagccgaggagaccctatccgagtttctttcaaaggccactggaacttctgtcgaatgggttcagatggttggaatgaagcctggtccggattctattggaatcatcgctgtttcacacaactgcagtggtgtggcagcacgtgcttgcggtcttgtgagcttagagcccacaaaggttgcggagattctcaaagcacgcccatcttggtatcgtgattgtaggtgcctaaatgtagcgacagtgatccctgccggaaatggagggaatattgagcttatatatatgcagacatatgcacctacaactcttgcagcggctcgagatttttggacactcagatacaccacaggtcttgaagatggaagtcttgtgatttgtgagaggtcgttgacttcttcaacaggtggccctactggcccccctgctctgaactttgttagggcagaaatgcttcctagtgggtaccttataagaccgtgcgagggtggtggctcaatgattcacattgttgatcatgttgattgggatgttatgagtgtgcccgaagttctaaggccactgtacgaatcaccaaagattatggcacataagatgacaatttctgcattacggtatttgaggcaaattgcacatgagacaagtggtgaatttgcttacaatagtggtcgtcaacccgctgttttgaggacttttagtcagaggttgagcagaggcttcaacgatgctataaatggattcatggatgatggatggtctttaatgagcagtgatggtggagatgatgtgacagtaactattaattcttcctcaagtaaacttgtgggttctgtgaattcatcccctctctttgcctttggtggtggtatcttgtgtgcaaaggcttctatgttgctccagaatgttccacctgctttacttgttcgttttctgagggaacatcgttctgagtgggctgaatgtggagtggatgcatattctcctgcatcacttagagacagctcccatcccatcttaggtgcgagagctggtggcagctttcatggcggtcaagttattctaccccttgcacaaactgtggaaaatgaagagttcttggaggtgatcagacttgaaggccatgaattttgccctgacaatgtagccagggacacttatctattgcagctgtgcaatggagttgatgagaatgctactggtgcatgctctcagcttgtttttgcccccattgatgaatcttttgcagataatgcccctttgcttccttctggttttcgtgtaatcccagttgaatctaaaacagaggtgcatgcagctgccagaactcttgaccttgcatctactttagaagtaggttcaggtggtgcgacgcgccccaacagtgagggtgcctcaactacatctaacttgagatctgtcttgactatagcattccaattcacatatgagaatcattatcgcgaaaatatggcagcaatggctcggcaatatgttcgcagcattgtggcttcagtacagagggttgccatggccatctctccttcacgcgtgggttcgcttggttcaaaacatttaactgggtcccctgagggtctcactttagcgcgctggatttcctgcagttacaagtttcatactggcgccgatctctttcgttccgagcccgatgtcgagcctcaagcgggtgattctttactgaagaccctgtggcattatcctgatgcaattatgtgctgcactctgaaggcgtcgcccatgtttacatttgcaaatcaggcaggccttgacatgctcgaaaccacactcatcgctcttcaggacataacgctcgagaagattctcaatgacagtggccgcaaggttttttgtactgaattctctaaaatcatgcagcagggatttgcttatctgcctggtggggtatgcctatcgagcatgggtaggcctgcatcttacgatcaggttgtcgcgtggaaggtccttgatgatgaggacactcatcattgcttggccttcatgttcatgaactggtcatttgtctaa

SEQ ID NO: 2 - protein, 850 aa, synthetic construct
MAMVGKEGKSQQHELQHIDAGKYVRYTPEQVEALERVYMECPKPSSLRRQQLIRECPILSNIEPKQIKVWFQNRRCREKQRKEASRLQTVNRKLSAMNKLLMEENDRLQKQVSHLVYENGCMRQQIHTPSATTTDTSCESVVTSGPHKQQQNTKLQHLQRDANNPAGLLAIAEETLSEFLSKATGTSVEWVQMVGMKPGPDSIGIIAVSHNCSGVAARACGLVSLEPTKVAEILKARPSWYRDCRCLNVATVIPAGNGGNIELIYMQTYAPTTLAAARDFWTLRYTTGLEDGSLVICERSLTSSTGGPTGPPALNFVRAEMLPSGYLIRPCEGGGSMIHIVDHVDWDVMSVPEVLRPLYESPKIMAHKMTISALRYLRQIAHETSGEFAYNSGRQPAVLRTFSQRLSRGFNDAINGFMDDGWSLMSSDGGDDVTVTINSSSSKLVGSVNSSPLFAFGGGILCAKASMLLQNVPPALLVRFLREHRSEWAECGVDAYSPASLRDSSHPILGARAGGSFHGGQVILPLAQTVENEEFLEVIRLEGHEFCPDNVARDTYLLQLCNGVDENATGACSQLVFAPIDESFADNAPLLPSGFRVIPVESKTEVHAAARTLDLASTLEVGSGGATRPNSEGASTTSNLRSVLTIAFQFTYENHYRENMAAMARQYVRSIVASVQRVAMAISPSRVGSLGSKHLTGSPEGLTLARWISCSYKFHTGADLFRSEPDVEPQAGDSLLKTLWHYPDAIMCCTLKASPMFTFANQAGLDMLETTLIALQDITLEKILNDSGRKVFCTEFSKIMQQGFAYLPGGVCLSSMGRPASYDQVVAWKVLDDEDTHHCLAFMFMNWSFV